(19) United States
(12) Patent Application Publication
(10) Pub. No.: US 2004/0241671 A1
(43) Pub. Date: Dec. 2, 2004

(54) Sequences involved in phenomena of tumour suppression, tumour reversion, apoptosis and/or virus resistance and their us as medicines

(76) Inventors: Adam Telerman, Paris (FR); Robert Amson, Paris (FR); Marius Tuijnder, Mechelen (BE); Laurent Susini, Montmorency (FR)

Correspondence Address: Pennie & Edmonds, 1667 K Street N W, Washington, DC, 20006, US

(21) Appl. No.: 10/466,894

(22) PCT Filed: Jan. 23, 2002
(86) PCT No.: PCT/FR02/00273

(51) Int. Cl. C12Q 1/68; C07H 21/04; C12P 21/02; C12P 19/34; C07K 14/525

(52) U.S. Cl. 435/6; 435/69.5; 435/320.1; 435/325; 530/351; 536/23.5

(57) ABSTRACT

This invention is directed to sequences that are involved in the molecular pathways of tumor suppression, tumor reversion, apoptosis and/or virus resistance. The use of the compounds having these sequences, or encoded by them, in treating cancer, neurodegenerative diseases or viral diseases, and screening methods for compounds having such therapeutic properties are also encompassed.

The present invention relates to the demonstration of genes involved in the molecular pathways of tumor suppression, tumor reversion, apoptosis and/or resistance to viruses.

The present invention was made possible by the isolation of cDNA corresponding to messenger RNAs expressed or suppressed during tumor suppression, tumor reversion and/or the process of apoptosis.

In order to isolate the genes activated or inhibited during tumor reversion, an overall combing of the gene expression in a malignant cell line (U937) and a derived cell line (US4) with suppression of the malignant phenotype has been carried out. Comparison of the genes expressed (RNA messengers expressed in the two cell types) has made it possible to demonstrate differentially expressed genes, i.e. genes expressed in one of the cells whereas they are not expressed in the other (the genes may be activated or inhibited).

It is readily deduced therefrom that these genes are at least involved in the cancerization process, in one case by their absence and, in the other case, by their presence.

For this differential study, the method used is the method described in 1992 by Liang and Pardee (Differential display of eukaryotic mRNA by means of a polymerase chain reaction).

In order to develop a model, the inventors have formed the following hypotheses: if it were possible to select, from a tumor which is sensitive to the cytopathic effect of the H-1 parvovirus, cells which were resistant, then this resistance might be due to a change in their malignant phenotype. It was possible to demonstrate this for US4 cells selected from U937 cancerous cells. Unlike the parental line U937, the US4 clones (and also the US3 clones which will not figure in the present invention) are resistant to the cytopathic effect of the H-1 parvovirus.

At the molecular level, it has been possible to observe that this suppression of the malignant phenotype goes together with an activation of expression of the p₂₁^{waf1} gene, independently of the expression of the p53 gene.

The approach to the problem according to the present invention has made it possible to isolate sequences directly linked to several precise functions. As a consequence, unlike the random sequencing of ESTs, the sequences are sequences whose function is known due to the fact that they are involved in the process of suppression of the malignant phenotype, of tumor reversion or of apoptosis and/or in resistance to viruses.

Tumor reversion differs from tumor suppression by the fact that it encompasses a broader domain than that of tumor suppressor genes. In other words, tumor reversion takes place through the implementation of metabolic and/or molecular pathways which are not limited to the metabolic molecular pathways in which tumor suppressor genes are involved.

Thus, the present invention relates in particular to novel sequences and also to the use of these sequences in diagnosis and for implementing methods for screening compounds to be tested. The invention also relates to methods for detecting and/or assaying the sequences of the invention or their expression product(s) in a biological sample.

The present invention relates first of all to an isolated nucleotide sequence comprising a nucleotide sequence chosen from the group comprising:

a) SEQ ID No. 1 to SEQ ID No. 2280,

b) a nucleotide sequence of at least 15 consecutive nucleotides of a sequence as defined in a),

c) a nucleotide sequence having a percentage identity of at least 80%, after optimal alignment, with a sequence defined in a) or b),

d) a nucleotide sequence which hybridizes, under high stringency conditions, with a sequence defined in a) or b), and

e) a complementary nucleotide sequence or the RNA sequence corresponding to a sequence as defined in a), b), c) or d).

The nucleotide sequence according to the invention defined in c) has a percentage identity of at least 80%, after optimal alignment, with a sequence as defined in a) or b) above, preferably of at least 90%, most preferably of at least 98%.

The terms “nucleotide sequence”, “nucleic acid”, “nucleic acid sequence”, “polynucleotide”, “oligo-nucleotide” or “polynucleotide sequence”, terms which will be used indifferently in the present description, are intended to denote a precise chain of nucleotides, which may or may not be modified, making it possible to define a fragment or a region of a nucleic acid, which may or may not comprise natural nucleotides, and possibly corresponding equally to a double-stranded DNA, a single-stranded DNA and transcription products of said DNAs. Thus, the nucleic acid sequences according to the invention also encompass PNAs (peptide nucleic acids), or the like.

The fragments of the nucleotide sequences of the invention comprise at least 15 consecutive nucleotides.

Preferentially, they comprise at least 20 consecutive nucleotides, and even more preferentially they comprise at least 30 consecutive nucleotides.

It should be understood that the present invention does not relate to the nucleotide sequences in their natural chromosomal environment, i.e. in the natural state. They are sequences which have been isolated and/or purified, i.e. they have been taken directly or indirectly, for example by copying, their environment having been at least partially modified. The nucleic acids obtained by chemical synthesis are also intended to be denoted.

For the purpose of the present invention, the term “percentage identity” between two nucleic acid or amino acid sequences is intended to denote a percentage of nucleotides of amino acid residues which are identical between the two sequences to be compared, obtained after best alignment, this percentage being purely statistical with the differences between the two sequences being distributed randomly and through their entire length. The term “best alignment” or “optimal alignment” is intended to denote the alignment for which the percentage identity determined as below is the highest. Sequence comparisons between two nucleic acid or amino acid sequences are traditionally carried out by comparing these sequences after having aligned them optimally, said comparison being carried out by segment or by “window of comparison” in order to identify and compare local regions of sequence similarity. The optimal alignment of the sequences for comparison can be carried out, besides manually, by means of the local homology algorithm of Smith and Waterman (1981), by means of the local homology algorithm of Neddleman and Wunsch (1970), by means of the similarity search method of Pearson and Lipman (1988), by means of computer software using these algorithms (GAP, BESTFIT, BLAST P, BLAST N, FASTA and TFASTA in the Wisconsin Genetics Software Package, Genetics Computer Group, 575 Science Dr., Madison, Wis.). In order to obtain the optimal alignment, the BLAST program is preferably used, with the BLOSUM 62 matrix. The PAM or PAM250 matrices can also be used.

The percentage identity between two nucleic acid or amino acid sequences is determined by comparing these two optimally aligned sequences, the nucleic acid or amino acid sequences to be compared possibly comprising additions or deletions with respect to the reference sequence for optimal alignment between these two sequences. The percentage identity is calculated by determining the number of identical positions for which the nucleotide or the amino acid residue is identical between the two sequences, dividing this number of identical positions by the total number of positions compared, and multiplying the result by 100 in order to obtain the percentage identity between these two sequences.

The expression “nucleic acid sequences having a percentage and identity of at least 80%, preferably of at least 90%, more preferably of at least 98%, after optimal alignment, with a reference sequence” is intended to denote the nucleic acid sequences exhibiting, compared to the reference nucleic acid sequence, certain modifications, such as in particular a deletion, a truncation, an extension, a chimeric fusion, and/or a substitution, in particular of the point type, and the nucleic acid sequence of which has at least 80%, preferably at least 90%, more preferably at least 98%, identity, after optimal alignment, with the reference nucleic acid sequence. Preferably, the specific or high stringency hybridization conditions will be such that they ensure at least 80%, preferably at least 90%, more preferably at least 98%, identity, after optimal alignment, between one of the two sequences and the sequence complementary to the other.

A hybridization under high stringency conditions means that the conditions of temperature and of ionic strength are chosen such that they allow the hybridization between two complementary nucleic acid fragments to be maintained. By way of illustration, high stringency conditions for the hybridization step for the purposes of defining the nucleotide sequences described above are advantageously as follows:

The DNA-DNA or DNA-RNA hybridization is carried out in two steps:

(1) prehybridization at 42° C. for 3 hours in phosphate buffer (20 mM, pH 7.5) containing 5×SSC (1×SSC corresponds to a solution of 0.15M NaCl +0.015M sodium citrate), 50% of formamide, 7% of sodium dodecyl sulfate (SDS), 10× Denhardt's, 5% of dextran sulfate and 1% of salmon sperm DNA; (2) hybridization per se for 20 hours at a temperature which depends on the length of the probe (i.e.: 42° C. for a probe >100 nucleotides in length) followed by two washes of 20 minutes at 20° C. in 2×SSC+2% SDS, and 1 wash of 20 minutes at 20° C. in 0.1×SSC+0.1% SDS. The final wash is carried out in 0.1×SSC+0.1% SDS for 30 minutes at 60° C. for a probe >100 nucleotides in length. The high stringency hybridization conditions described above for a polynucleotide of defined length can be adapted by those skilled in the art for longer or shorter oligonucleotides, according to the teaching of Sambrook et al., 1989.

Among the nucleotide sequences having a percentage identity of at least 80%, preferably of at least 90%, more preferably of at least 98%, after optimal alignment, with the sequences according to the invention, preference is also given to the nucleic acid sequences which are variants of the sequences of the invention, or of their fragments, i.e. all of the nucleic acid sequences corresponding to allelic variants, i.e. individual variations of the sequences of the invention.

The term “variant nucleotide sequence” is intended to denote any RNA or cDNA resulting from a mutation and/or variation of a splice site of the genomic DNA corresponding to the nucleotide sequences of the invention.

A subject of the present invention is also a polypeptide encoded by a nucleotide sequence in accordance with the invention.

For the purpose of the present invention, the term “polypeptide” is intended to denote equally proteins or peptides.

According to a particular embodiment, the polypeptides in accordance with the invention comprise a polypeptide chosen from:

a) a polypeptide encoded by a nucleotide sequence in accordance with the invention,

b) a polypeptide having at least 80% identity with a polypeptide as defined in a),

c) a fragment of at least 5 amino acids of a polypeptide as defined in a) or b),

d) a biologically active fragment of a polypeptide as defined in a)., b), or c), and

e) a modified polypeptide of a polypeptide defined in a), b), c) or d).

The evaluation of the percentage identity is understood to be after optimal alignment of the sequences concerned. The expression “polypeptide the amino acid sequence of which has a percentage identity of at least 80%, preferably of at least 90%, more preferably of at least 98%, after optimal alignment, with a reference sequence” is intended to denote the polypeptides exhibiting certain modifications compared to the reference polypeptide, such as in particular one or more deletion(s) or truncation(s), an extension, a chimeric fusion and/or one or more substitution(s).

Among the polypeptides the amino acid sequence of which has a percentage identity of at least 80%, preferably of at least 90%, and more preferably of at least 98%, after optimal alignment, with a reference sequence such as a polypeptide in accordance with the present invention, or with one of its fragments, preference is given to the variant polypeptides encoded by the variant nucleotide sequences as previously defined, in particular the polypeptides the amino acid sequence of which exhibits at least one mutation corresponding in particular to a truncation, deletion, substitution and/or addition of at least one residue compared to the polypeptide sequences of the invention or to one of their fragments.

The present invention also relates to a cloning and/or cellular expression vector, characterized in that it comprises a nucleotide sequence according to the invention or in that it encodes a polypeptide according to the invention. Such a vector can also contain the elements required for the expression and, optionally, the secretion of the polypeptide in a host cell. Such a host cell is also the subject of the present invention.

The vectors comprising promoter and/or regulatory sequences are also part of the present invention. Said vectors preferably comprise a promoter, translation initiation and termination signals, and also the appropriate regions for regulating transcription. It must be possible for them to be maintained stably in the cell, and they can also possess specific signals able to permit secretion of the translated protein.

These various control signals are chosen as a function of the cellular host used. To this effect, the nucleic acid sequences according to the invention can be inserted into vectors which replicate autonomously in the chosen host or vectors which integrate in the chosen host.

Among the systems which replicate autonomously, use is preferably to be made, depending on the host cell, of systems of the plasmid or viral type, the viral vectors possibly being in particular adenoviruses (5), retroviruses, lentiviruses, poxviruses or herpesviruses (5a). Those skilled in the art are aware of the technology which can be used for each of these systems.

Advantageously, the vectors in accordance with the invention comprise a sequence for tissue-specific targeting and/or expression.

When integration of the sequence into the chromosomes of the host cell is desired, use may be made, for example, of systems of the plasmid or viral type; such viruses are, for example, retroviruses (6) or AAVs (7).

Among the nonviral vectors, preference is given to naked polynucleotides such as the naked DNA or the naked RNA according to the technique developed by the company VICAL, bacterial artificial chromosomes (BACs), yeast artificial chromosomes (YACs) for expression in yeast, mouse artificial chromosomes (MACs) for expression in murine cells and, preferably, human artificial chromosomes (HACs) for expression in human cells.

Such vectors are prepared according to the methods commonly used by those skilled in the art, and the clones resulting therefrom can be introduced into a suitable host by standard methods, such as, for example, lipofection, electroporation, heat shock, transformation after chemical permeabilization of the membrane, or cell fusion.

The invention also comprises the host cells, in particular the eukaryotic and prokaryotic cells, transformed with the vectors according to the invention and also the transgenic animals, preferably the mammals, except humans, comprising one of said transformed cells according to the invention. These animals can be used as models, for studying the etiology of inflammatory and/or immune diseases, and in particular inflammatory diseases of the digestive tract, or for studying cancers.

Among the cells which can be used for the purposes of the present invention, mention may be made of bacterial cells (8), but also yeast cells (9), along with animal cells, in particular mammalian cell cultures (10), and in particular Chinese hamster ovary (CHO) cells. Mention may also be made of insect cells in which it is possible to use methods using, for example, baculoviruses (11). A preferred cellular host for expression of the proteins of the invention consists of COS cells.

Among the mammals according to the invention, animals such as rodents, in particular mice, rats or rabbits, expressing a polypeptide according to the invention, are preferred.

These transgenic animals are obtained, for example, by homologous recombination on embryonic stem cells, transfer of these stem cells to embryos, selection of the chimeras affected in the reproductive lines, and growth of said chimeras.

The transgenic animals according to the invention can thus overexpress the gene encoding the protein according to the invention, or their homologous gene, or express the gene into which a mutation is introduced. These transgenic animals, in particular mice, are obtained, for example, by transfection of a copy of this gene under the control of a strong ubiquitous promoter, or a promoter selective for a tissue type, or after viral transcription.

The cells of mammals according to the invention can be used in a method for producing a polypeptide according to the invention, as described below, and can also be used as an analytical model.

The cells or mammals transformed as described above can also be used as models in order to study the interactions between the polypeptides according to the invention, and the chemical or protein compounds involved directly or indirectly in the activities of the polypeptides according to the invention, in order to study the various mechanisms and interactions which come into play.

They can in particular be used to select products which interact with the polypeptides according to the invention, or their [lacuna], as a cofactor or as an inhibitor, in particular a competitive inhibitor, or which have an agonist or antagonist activity with respect to the activity of the polypeptides according to the invention. Preferably, said transformed cells or transgenic animals are used as a model, in particular for selecting products for combating pathological conditions associated with abnormal expression of this gene.

A subject of the present invention is also a monoclonal or polyclonal antibody, a fragment of this antibody or a chimeric antibody capable of specifically recognizing a polypeptide in accordance with the present invention.

The specific monoclonal antibodies can be obtained according to the conventional method of hybridoma culture well known to those skilled in the art.

The antibodies according to the invention are, for example, humanized antibodies, or Fab or F(ab′)² fragments. They may also be in [lacuna] of immunoconjugates or of antibodies which are labeled in order to obtain a detectable and/or quantifiable signal.

The antibodies in accordance with the invention, but also the immunoconjugates, are therefore capable of specifically recognizing a polypeptide according to the present invention.

The specific polyclonal antibodies can be obtained from the serum of an animal immunized against polypeptides of the invention, in particular produced by genetic recombination or by peptide synthesis, according to usual procedures.

The advantage of antibodies which specifically recognize the polypeptides, their variants or their immunogenic fragments, according to the invention, is in particular noted.

A subject of the invention is also the use of a nucleotide sequence in accordance with the present invention, as a probe or primer for detecting, identifying, assaying and/or amplifying nucleic acid sequences.

According to the invention, the nucleotide sequences which can be used as a probe or as a primer in methods for detecting, identifying, assaying and/or amplifying nucleic acid sequences are a minimum of 15 bases, preferably of 20 bases, or better still of 25 to 30 bases, in length.

The probes and primers according to the invention can be labeled directly or indirectly with a radioactive or nonradioactive compound by methods well known to those skilled in the art, in order to obtain a detectable and/or quantifiable signal.

The nucleic acid sequences according to the invention which are unlabeled can be used directly as a probe or primer.

The sequences are generally labeled in order to obtain sequences which can be used for many applications. The primers or the probes according to the invention are labeled with radioactive elements or with nonradioactive molecules.

Among the radioactive isotopes used, mention may be made of ³²P, ³³P, ³⁵S³H or ¹²⁵I. The nonradioactive entities are selected from ligands such as biotin, avidin, streptavidin or digoxigenin, haptens, dyes, and luminescent agents such as radioluminescent, chemiluminescent, bioluminescent, fluorescent or phosphorescent agents.

The nucleotide sequences according to the invention can thus be used as a primer and/or probe in methods using the PCR (polymerase chain reaction) technique (11a). This technique requires choosing pairs of oligonucleotide primers which frame the fragment which must be amplified. Reference may, for example, be made to the technique described in U.S. Pat. No. 4,683,202. The amplified fragments can be identified, for example after agarose or polyacrylamide gel electrophoresis, or after a chromatographic technique such as gel filtration or ion exchange chromatography, and then sequenced. The specificity of the amplification can be controlled using, as primers, the nucleotide sequences of the invention and, as matrices, plasmids containing these sequences or else the derived amplification products. The amplified nucleotide fragments can be used as reagents in hybridization reactions in order to demonstrate the presence, in a biological sample, of a target nucleic acid of sequence complementary to that of said amplified nucleotide fragments.

The invention is also directed toward the nucleic acids which can be obtained by amplification using primers according to the invention.

Other techniques for amplifying the target nucleic acid can advantageously be employed as an alternative to PCR (PCR-like), using a pair of primers of nucleotide sequences according to the invention. The term “PCR-like” is intended to denote all methods using direct or indirect reproductions of nucleic acid sequences, or else in which the labeling systems have been amplified; these techniques are of course known. In general, it involves amplification of the DNA with a polymerase; when the sample of origin is an RNA, it is advisable to perform a reverse transcription beforehand. A large number of methods for this amplification currently exist, such as, for example, the SDA (strand displacement amplification) technique (12), the TAS (transcription-based amplification system) technique described by (13), the 3SR (self-sustained sequence replication) technique described by (14), the NASBA (nucleic acid sequence based amplification) technique described by (15), the TMA (transcription mediated amplification) technique, the LCR (ligase chain reaction) technique described by (16), the RCR (repair chain reaction) technique described by (17), the CPR (cycling probe reaction) technique described by (18), and the Q-beta-replicase amplification technique described by (19). Some of these techniques have since been improved.

When the target polynucleotide to be detected is an mRNA, an enzyme of the reverse transcriptase type is advantageously used, prior to carrying out an amplification reaction using the primers according to the invention or to carrying out a method of detection using the probes of the invention, in order to obtain a cDNA from the mRNA contained in the biological sample. The cDNA obtained will then serve as a target for the primers or the probes used in the method of amplification or of detection according to the invention.

The probe hybridization technique can be carried out in various ways (20). The most general method consists in immobilizing the nucleic acid extracted from the cells of various tissues or from cells in culture, on a support (such as nitrocellulose, nylon or polystyrene) and in incubating, under well-defined conditions, the immobilized target nucleic acid with the probe. After hybridization, the excess probe is removed and the hybrid molecules formed are detected by the appropriate method (measurement of the radioactivity, of the fluorescence or of the enzyme activity associated with the probe).

According to another embodiment of the nucleic acid probes according to the invention, the latter can be used as capture probes. In this case, a probe, termed “capture probe” is immobilized on a support and is used to capture, by specific hybridization, the target nucleic acid obtained from the biological sample to be tested, and the target nucleic acid is then detected using a second probe, termed “detection probe”, which is labeled with a readily detectable element.

The nucleotide sequences according to the invention can, moreover, be of value when they are used as antisense nucleotides, i.e. nucleotides whose structure provides, by hybridization with the target sequence, inhibition of the expression of the corresponding product. They can also be used as sense nucleotides which, by interaction with proteins involved in regulating the expression of the corresponding product, will induce either an inhibition or an activation of this expression.

A subject of the invention is also the use of a nucleotide sequence according to the present invention, for producing or synthesizing a recombinant polypeptide.

The method for producing a polypeptide of the invention in recombinant form, which is itself included in the present invention, is characterized in that the transformed cells, in particular the cells or mammals of the present invention, are cultured under conditions which allow expression of a recombinant polypeptide encoded by a nucleotide sequence according to the invention, and said recombinant polypeptide is recovered.

The recombinant polypeptides, characterized in that they can be obtained using said method of production, are also part of the invention.

The recombinant polypeptides obtained as indicated above can be in both glycosylated and unglycosylated form, and may or may not have the natural tertiary structure.

The sequences of the recombinant polypeptides can also be modified in order to improve their solubility, in particular in aqueous solvents.

Such modifications are known to those skilled in the art, such as, for example, the deletion of hydrophobic domains or the substitution of hydrophobic amino acids with hydrophilic amino acids.

These polypeptides can be produced from the nucleic acid sequences defined above, according to the techniques for producing recombinant polypeptides known to those skilled in the art. In this case, the nucleic acid sequence used is placed under the control of signals which allow its expression in a cellular host.

An efficient system for producing a recombinant polypeptide needs to have a vector and a host cell according to the invention.

These cells can be obtained by introducing into host cells a nucleotide sequence inserted into a vector as defined above, and then culturing said cells under conditions which allow the replication and/or expression of the transfected nucleotide sequence.

The methods used for the purification of a recombinant polypeptide are known to those skilled in the art. The recombinant polypeptide can be purified from cell lysates and extracts, or from the culture medium supernatant, by methods used individually or in combination, such as fractionation, chromatography methods, immunoaffinity techniques using specific monoclonal or polyclonal antibodies, etc.

The polypeptides according to the present invention can also be obtained by chemical synthesis using one of the many known forms of peptide synthesis, for example the techniques using solid phases (21) or techniques using partial solid phases, by fragment condensation or by conventional synthesis in solution.

The polypeptides which are obtained by chemical synthesis and which can comprise corresponding unnatural amino acids are also included in the invention.

The subject of the present invention is also a DNA chip, characterized in that it contains at least one nucleotide sequence in accordance with the present invention.

Specifically, the nucleotide sequences according to the invention intended to be used as a probe or as a primer for detecting, identifying, assaying and/or amplifying nucleic acid sequences can be immobilized covalently or noncovalently on a support, this support being a DNA chip or a high density filter.

The term “DNA chip” or “high density filter” is intended to denote a support to which are attached DNA sequences, each one of them being able to be pinpointed by its geographical location. These chips or filters differ mainly by their size, the material of the support and, optionally, the number of sequences which are attached thereto.

In particular, an in situ synthesis can be carried out by photochemical addressing or by inkjet. Other techniques consist in carrying out an ex situ synthesis and attaching the probes to the support of the DNA chip by mechanical or electrical addressing or by ink-jet. These various methods are well known to those skilled in the art.

A subject of the invention is also a protein chip comprising a polypeptide or an antibody according to the invention.

Such a protein chip makes it possible to study the interactions between the polypeptides according to the invention and other proteins or chemical compounds, and can thus be of use for screening compounds which interact with the polypeptides according to the invention.

The protein chips according to the invention can also be used to detect the presence of antibodies directed against the polypeptides according to the invention in the serum of patients to be tested. A protein chip comprising an antibody according to the invention can also be used to detect, this time, the presence of polypeptides, in the serum of patients, which can be recognized by said antibody.

A subject of the present invention is also the use of the compound chosen from a nucleotide sequence, a polypeptide, a vector, a cell or an antibody according to the invention, for preparing a medicinal product.

The pathological conditions more specifically targeted are viral diseases and diseases which are characterized by the development of tumor cells or cellular degeneration, such as Alzheimer's disease or schizophrenia. Thus, the abovementioned medicinal product is intended for the prevention and/or treatment of these diseases. In particular, the disease targeted is cancer.

One of the advantages of the present invention is that it has demonstrated the involvement of a large number of nucleotide sequences in the phenomena of tumor suppression, tumor reversion, apoptosis and/or viral resistance. These sequences are therefore differentially expressed when one of these abovementioned processes is set in motion. Consequently, in the presence of a patient in whom the triggering of one of these processes is suspected, or for whom it is desired to verify the absence of such a triggering, it is of use to be able to determine, or even quantify, the expression of one or more sequence(s) in accordance with the invention using a biological sample from said patient. Optionally, the analysis of the expression of one or more of said sequences can be accompanied by a comparison with a reference level of expression corresponding to that of a healthy individual.

Consequently, the invention therefore also comprises a method for the diagnosis and/or for the prognostic evaluation of a viral disease or a disease characterized by the development of a tumor or cellular degeneration, comprising analysis of the expression of at least one sequence of the invention using a biological sample from a patient to be tested.

According to a preferred embodiment, said method comprises the following steps:

isolating the messenger RNA from a biological sample derived from a patient to be tested,

preparing the complementary cDNA from said messenger RNA,

optionally, amplifying a portion of complementary DNA corresponding to at least one sequence of the invention, and

detecting the complementary DNA possibly amplified.

In particular, the analysis of the expression of the sequence can be carried out using a DNA chip as described above.

Among the sequences of the present invention, some have the characteristic of being receptors expressed at the surface of the cells and, in order to understand the mechanism thereof in the context of the above-mentioned processes, it is advantageous to search for compounds capable of interacting with this receptor, i.e. of interacting with a polypeptide in accordance with the invention; it is also necessary to provide for the secreted protein. This also applies to the polypeptides in accordance with the invention corresponding to secreted proteins (at the surface or outside the cells), hormone-like proteins, etc. Consequently, a subject of the present invention is also a method for screening compounds capable of attaching to a peptide in accordance with the invention, comprising the following steps:

bringing a polypeptide or a cell according to the invention into contact with a candidate compound, and

detecting the formation of a complex between said candidate compound and said polypeptide or said cell.

A method for screening compounds can also be advantageous relative to compounds capable of interacting with a nucleotide sequence according to the invention, or even with the sequences required for the expression or the regulation of these sequences.

Specifically, the compounds are capable of interacting with said sequences, with the effect of reducing, inhibiting or, on the contrary, potentiating the expression of the sequences in question. Such a method comprises the following steps:

bringing a nucleotide sequence or a cell according to the invention into contact with a candidate compound, and

detecting the formation of a complex between said candidate compound and said nucleotide sequence or said cell.

For reasons mentioned above, it may therefore be advantageous to search for and/or to assay, in a biological sample or specimen from a patient to be tested, the presence of a nucleotide sequence according to the invention. Such a detection and/or assaying method comprises the following steps:

bringing a nucleotide sequence according to the invention, which is labeled, into contact with the biological sample to be tested, under the conditions required for the formation of a hybrid, and

detecting and/or assaying the hybrid possibly formed between said nucleotide sequence and the nucleic acid present in said biological sample.

This method may also comprise a step for amplifying the nucleic acid of said biological sample using primers chosen from the nucleotide sequences according to the invention.

In particular, this method can be carried out using the DNA chip described above.

Those skilled in the art are able to implement such a method and can in particular use a reagent kit comprising:

a) a nucleotide sequence according to the invention used as a probe,

b) the reagents required to carry out a hybridization reaction between said probe and the nucleic acid of the biological sample,

c) the reagents required to detect and/or assay the hybrid formed between said probe and the nucleic acid of the biological sample.

Such a kit may also contain positive or negative controls in order to ensure the quality of the results obtained.

Similarly, in the context of the present invention, it is also possible to envision detecting and/or assaying a polypeptide according to the invention, and this method therefore comprises the following steps:

bringing the biological sample into contact with a labeled antibody according to the invention, and

detecting and/or assaying the complex formed by said antibody of polypeptide present in said sample.

Advantageously, this method can be carried out using the protein chip as described above. Here again, those skilled in the art are able to carry out such a method, and can in particular use a reagent kit comprising:

a) a monoclonal or polyclonal antibody according to the invention;

b) optionally, reagents for constituting a medium suitable for the antigen/antibody reaction;

c) the reagents for detecting the antigen/antibody complex.

Finally, a subject of the present invention is also a computer-readable medium or a computer medium on which at least one nucleotide sequence as claimed in claim 1 and/or at least one polypeptide sequence as defined in claim 3 or 4 are recorded. In particular, this medium is chosen from the group comprising:

a) a disk,

b) a hard disk,

c) a random access memory (RAM),

d) a read-only memory (ROM),

e) a CD-ROM.

The invention is not limited to the present description, but, on the contrary, it encompasses all the variants and will be understood more clearly in light of the experimental data below.

### FIGURES

FIG. 1 represents a tumor growth curve for the groups U937 and US4 in SCID mice.

FIG. 2 represents a curve of body weight of the mice carrying a U937 or US4 tumor.

1-Data Relating to the U937 and US4 Cells

As seen above, the present invention uses the parental cells U937 and the “derived” cells US4. In fact, US4 cells and US3 cells (which do not figure in the context of the present invention) share certain characteristics. The method for obtaining them and also their properties are reported below.

Selection and Characterization of US3 and US4 Cells

U937 cells were subjected to two series of limiting dilution until a single clonal population was obtained. These cells were infected with the H-1 parvovirus. The cytopathic effect of the virus creates massive cell death, sparing two resistant clones, namely US3 and US4, after three months of continuous culturing. The survival of the cells is defined as the relative number of viable cells in the culture infected with the H-1 virus compared to the untreated culture, as measured four days after reinfection. To measure the tumorigenicity, 107 U937, US3 or US4 cells were injected subcutaneously into scid/scid mice (4 or 5 weeks old). The tumorigenicity is expressed by the number of tumors developed by the mice during the two months following the injection.

The approach was as follows: from clonal populations of malignant cells, subclones with a suppressed tumorigenic phenotype were derived. This selection, done via the H-1 parvovirus, is produced by elimination of the tumor cells, which are preferentially killed, while at the same time sparing the normal cells. The selection of cells resistant to the cytopathic effect of the H-1 parvovirus outside of a sensitive tumor can produce cells which have a decreased malignant phenotype.

On this basis, a clonal population of U937 cells was isolated, these cells are sensitive to the cytopathic effect of the H-1 parvovirus and, as regards the US3 and US4 clones, they are resistant to the virus. The US3 and US4 clones have a strong suppressed tumorigenic phenotype while the parental U937 cells develop tumors in 80% of cases among the scid/scid mice having been infected with the parvovirus; the US3 cells form only one tumor and the US4 cells develop one tumor per 20 inoculations with 10⁷ cells. The results are given in the table below.

Resistance to the H-1 Parvovirus and Tumorigenicity of the U937, US3 and US4 Cells

| | Survival of cells after | Tumorigenicity in |
|---|---|---|
| Cell lines | infection with the H-1 virus | scid/scid mice |
| U937 | 0.4 | 16/20 |
| US3 | 96 | 0/10 |
| US4 | 89 | 1/20 |

2-Materials and Methods

This involves comparing the growth of subcutaneous tumors in SCID mice, induced by subcutaneous injection of transfected U937 and US4 human leukemia cell lines.

A. Leukemia Cell Lines and Culturing Conditions

All the injected cells of the U937 (ATCC) and US4 cell lines were provided in the form of cell suspensions in flasks filled with RPMI-1640 culture medium supplemented with 2 mM L-glutamine, 10% fetal bovine serum and gentamycin.

The U937 cell line is a CD4+ human monocyte cell line derived from a patient with a diffuse histiocytic lymphoma (1).

The cells were counted in a hemocytometer and their viability was tested with 0.25% trypan blue dye exclusion. The viability was respectively 95.5% and 90.5% for the U937 cells and the US4 cells. The U937 and US4 cells were centrifuged and then resuspended in an RPMI medium, before being injected into SCID mice.

B. Animals

10 female SCID mice in good health (CB17/IcrHsd), 31 weeks old and weighing between 20 and 25 g, were supplied by Harlan France (Gannat, France). The animals were observed for 7 days in specific premises belonging to the Applicant, which is the specific pathogen free (SPF) animal care unit, before they were treated. The animal care unit (INRA, Dijon, France) is authorized by the French Ministers for Agriculture and for Research (approval no. A21100). The animal experiments were carried out according to the European guidelines on ethics regarding animal experiments (2) and the United Kingdom guidelines for welfare of animals in experimental neoplasia (3).

B.1. Environment

The animals were kept in rooms under controlled conditions with temperature (24±1° C.), humidity (55±1%), light period (12 h light/12 h dark) and air renewal. The animals were kept under SPF conditions and the temperature and humidity of the room were continually monitored. The ventilation system was programmed to give 14 renewals of air per hour without recirculation. Fresh air coming from the outside passes through a series of filters before being diffused evenly into each room. A high pressure (2 mm) was maintained in the experimentation rooms in order to prevent contamination or diffusion of pathogens within a mouse colony. All the personnel working under the SPF conditions follow the specific guidelines in consideration of hygiene and clothing when they enter the animal-rearing area.

B.2. Animal-Rearing

The animals were housed in polycarbonate cages (UAR, Epinay sur Orge, France) which were equipped so as to provide them with food and water. The standard size of the cages used is 637 cm² for 10 mice according to the standard internal operating procedures. The animal litter is made up of sterile wood shavings (UAR), which are replaced twice a week.

B.3. Feed and Drink

The animal feed was purchased from Extralabo (Provins, France). The feed was supplied ad libitum and was placed on the metal cover at the top of the cage. The water was also provided ad libitum, from water bottles equipped with rubber taps. The water bottles were washed, sterilized and replaced once a week. The water supply was sterilized by filtration with an absolute filter of 0.2 μm.

B.4. Animal and Cage Identification

After random distribution, the animals were identified with two different numbers tattooed on both ears. Each cage was labeled with a specific code

C—Experimental Data and Treatments

C.1. Induction of Tumors in the SCID Mice

Before the injection of cells, the SCID mice were distributed randomly in two groups, in a proportion of 5 mice per group. 10⁷ US4 or UP37 tumor cells in 0.2 ml of RPMI medium were inoculated subcutaneously into SCID mice, at time 0, for each point of injection. Each animal was given four injections of tumor cells located in different-regions; one in each flank and one in each shoulder.

C.2. Tumor Collection

When the tumors reached a volume of 1 500 mm², the mice were killed and the tumors were collected, weighed, frozen in liquid nitrogen, stored at −80° C., and then specifically labeled.

C.3. Control of Mice

The isoflurane forene (Minerve, Bondouble, France) was used to anesthetize the animals before the injection of cells for the sacrifice. After the injection of tumor cells, the mice were observed for 5 hours. The viability, behavior and body weight of the mice and the growth of the subcutaneous tumor were recorded twice a week.

During the experiment, the animals were killed, under anesthesia with isoflurane, by cervical dislocation if one of the following signs appeared:

sign of suffering (cachexia, weakening, difficulty in moving or in eating),

tumor growth up to 10% of body weight,

tumor ulceration and persistent exposure,

position of the tumor interfering with movement and/or feeding,

20% weight loss for three consecutive days.

An autopsy was carried out for each animal, in order to detect the presence of possible metastases or of morphological abnormalities.

D—Presentation of the Data

D.1. Survival Parameters

The calculation for the median and mean survival time was expressed as follows:

Mean survival time=S₁/(S₂−NT)

With: S1=the sum of the daily survivors from day 0 up to the end of the experiment (without the survivors “not taken into account”*)

S2=the number of animals at the start

NT=the number of animals “not taken into consideration”*

* “not taken into consideration”: these are animals with tumors smaller than the predetermined limit, considered to result from a deficient implantation of the tumor.

D.2. Tumor Inhibition System

The tumor size was measured twice a week with a pair of compasses and the tumor volume (in mm₃) is estimated according to the formula: (length×width²)/2 (4). The experiments were stopped when the tumor sizes in the mice reached 1 500 mm³.

After sacrifice, the tumors were excised and weighed.

The curve of tumor growth for the US4 and U937 groups was plotted using the mean of the tumor volumes.

The tumor doubling time for the US4 and U937 groups was defined as the amount of time required to reach a mean tumor volume of 200% during the growth period.

The specific growth period over one or two doubling times (TD) from cell injections is defined as follows:

specific growth period=(TD US4−TD U937)/TD U937

the growth period was calculated as the difference in median growth time of the US4 group and of the U937 group to reach the same tumor size.

D.3. Statistical Tests

All the statistical analyses were carried out with the StatView® software (Abacus concept, Berkeley, USA). The statistical analyses of the mean body weight changes, of the tumor doubling time and of the time to reach “V” were carried out using the Bonferroni/Dunn test. A value p<0.05 was considered to be significant. All the groups were compared with one another.

E—Results

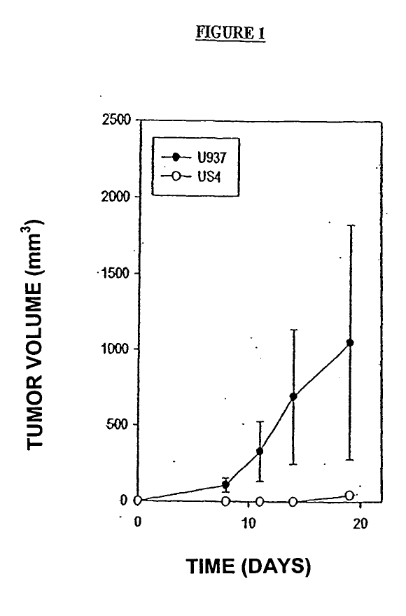

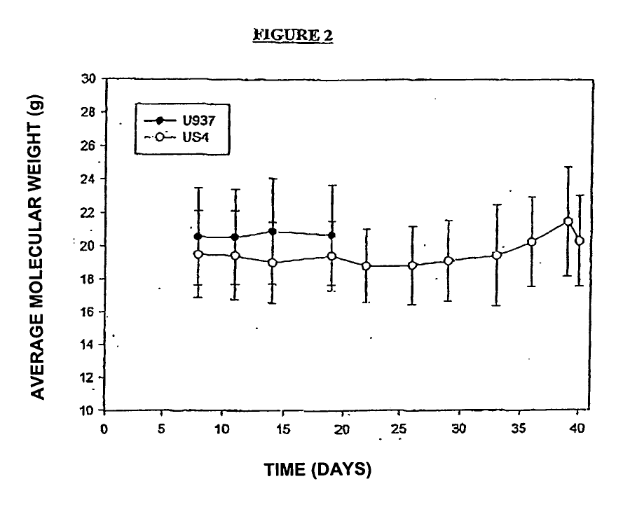

The curves of tumor volumes and mean body weights are shown in FIGS. 1 and 2 respectively.

No significant loss of body weight of the SCID mice, based on the two groups, was observed between day 8 and day 19.

A significant difference is observed between the two groups of SCID mice, regarding the time to reach “V”, whereas no significant difference was observed for the change in mean body weight (day 19-day 8) and for the doubling time.

The autopsies performed did not show the presence of metastases or of suspicious node development. Tumor collection was carried out on the animals sacrificed after anesthesia and cervical dislocation. The tumors were immediately placed in tubes, frozen in liquid nitrogen and stored at −80° C. The excised tumors were ovoid in shape, had a moderate consistency and were pinkish in color. The interactions of the tumor with its environment (skin and muscle tissue) was limited and superficial.

F—Conclusions

The US4 cell line showed a significantly lower rate of taking hold compared to the U937 cell line in the SCID mice.

The delay in growth between the US4 and U937 tumors was 23.5 days and the doubling time was equivalent.

### REFERENCES

(1) Sundström C. et Nilsson K., Int. J. Cancer, 17, 565, 1976.

(2) Principe d'éthique de l'expérimentation animale [Principles of ethics for animal experimentation], EEC Directive No. 86/609 of Nov. 24, 1986, Decree No. 87/848 of Oct. 19, 1987, Order of Application of Apr. 19, 1988.

(3) United Kingdom co-coordinating committee on cancer research guidelines for welfare of animals in experimental neoplasia, Br. J. Cancer, 77: 1-10, 1998.

(4) Bissery M. C. et al., Bull. Cancer, 78: 587, 1991.

(5) Perricaudet et al. (1992). La Recherche 23: 471.

(5a) Epstein (1992) Médecine/Sciences, 8, 902.

(6) Temin, (1986) Retrovirus vectors for gene transfer. In Kucherlapati R., ed. Gene.

(7) Carter, (1993) Curr. Op. Biotechnology 3, 533.

(8) Olins and Lee (1993), Curr. Op. Biotechnology 4: 520.

(9) Buckholz, (1993), Curr. Op. Biotechnology 4, 538.

(10) Edwards and Aruffo (1993), Curr. Op. Biotechnology, 4, 558.

(11) Luckow (1993), Curr. Op. Biotechnology 4, 564.

(11a) Rolfs, A. et al. (1991), Berlin: Springer-Verlag.

(12) Walker (1992), Nucleic Acids Res. 20: 1691.

(13) Kwoh, et al. (1989), Proc. Natl. Acad. Sci. USA, 86, 1173.

(14) Guatelli et al. (1990), Proc. Natl. Acad. Sci. USA 87: 1874.

(15) Kievitis et al. (1991), J. Virol. Methods, 35, 273.

(16) Landegren et al. (1988) Science 241, 1077.

(17) Segev, (1992), Kessler C. Springer Verlag, Berlin, N.Y., 197-205.

(18) Duck et al. (1990), Biotechniques, 9, 142.

(19) Miele et al. (1983), J. Mol. Biol., 171, 281.

(20) Matthews et al. (1988), Anal. Biochem., 169, 1-25.

(21) Stewart and Yound (1984), solid phase peptide synthesis, Pierce Chem. Company, Rockford, 111, 2nd ed., (1984).

1. An isolated nucleotide sequence comprising a nucleotide sequence chosen from the group comprising:
a) SEQ. ID NO. 1 to SEQ. ID NO. 2280,
b) a nucleotide sequence of at least 15 consecutive nucleotides of a sequence as defined in a),
c) a nucleotide sequence having a percentage identity of al least 80%, after optimal alignment, with a sequence defined in a) or b),
d) a nucleotide sequence which hybridizes, under high stringency conditions, with a sequence defined in a) or b), and
e) a complementary nucleotide sequence or the RNA sequence corresponding to a sequence as defined in a), b), c) or d).

2-30. (Canceled)
